SEQUENCE LISTING (2 sequences)

SEQ ID NO: 1 - DNA, 1377 bp, homo sapiens
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 458 aa, homo sapiens
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2005/0281822 A1
(43) Pub. Date: Dec. 22, 2005

(54) Method of administering and using VEGF inhibitors for the treatment of malignant pleural effusion

(76) Inventors: Jesse M. Cedarbaum, Larchmont, NY (US); Christopher G. Azzoli, New York, NY (US); Mark G. Kris, New York, NY (US); Jakob Dupont, Pelham, NY (US)

Correspondence Address: REGENERON PHARMACEUTICALS, INC, 777 OLD SAW MILL RIVER ROAD, TARRYTOWN, NY, 10591, US

(21) Appl. No.: 11/155,269

(22) Filed: Jun. 17, 2005

Related U.S. Application Data
(60) Provisional application No. 60/580,893, filed on Jun. 18, 2004.

(51) Int. Cl. A61K 39/395; A61K 38/18

(52) U.S. Cl. 424/145.1; 514/2

(57) ABSTRACT

Methods for treating a human patient suffering from malignant pleural effusion by administering an effective amount of a vascular endothelial growth factor (VEGF) inhibitor to the human patient. The VEGF inhibitor is a VEGF trap protein comprising a dimeric protein having two fusion polypeptides having the sequence of SEQ ID NO:2.

### RELATED APPLICATIONS

This application claims the benefit under 35 USC 119(e) to U.S. Ser. No. 60/580,893 filed 18 Jun. 2004, which application is incorporated by reference in its entirety.

### FIELD OF THE INVENTION

The invention relates to methods of treating patients with malignant pleural effusion (MPE). More specifically, the invention relates to methods of treating patients with MPE due to advanced non-small cell lung cancer (NSCLC), breast cancer, lymphoma, leukemia or mesothelioma.

### DESCRIPTION OF RELATED ART

Vascular endothelial growth factor (VEGF) expression is nearly ubiquitous in human cancer, consistent with its role as a key mediator of tumor neoangiogenesis. Blockade of VEGF function, by binding to the molecule or its VEGFR-2 receptor, inhibits growth of implanted tumor cells in multiple different xenograft models (see, for example, Gerber et al. (2000) Cancer Res. 60:6253-6258). A soluble VEGF antagonist, termed a “VEGF trap” or “VEGFR1R2 trap” has been described (Kim et al. (2002) Proc. Natl. Acad. Sci. USA 99:11399-404; Holash et al. (2002) Proc. Natl. Acad. Sci. USA 99:11393-8), which application is herein specifically incorporated by reference.

### BRIEF SUMMARY OF THE INVENTION

In a first aspect, the invention features a method of treating a human patient suffering from malignant pleural effusion, comprising administering a therapeutically effective amount of a vascular endothelial growth factor (VEGF) trap antagonist to the human patient. VEGF trap protein antagonists are described in WO 00/75319, herein specifically incorporated by reference.

According to the present invention, the VEGF trap protein antagonist is a fusion protein comprising immunoglobulin (Ig)-like domain components from two different VEGF receptor proteins fused to a multimerizing component. More specifically, the VEGF trap protein antagonists of the invention comprise a dimer of two fusion polypeptides, each polypeptide comprising an immunoglobulin (Ig)-like domain 2 of a Flt-1 and an Ig-like domain 3 of Fltk-1 or Flt-4 and a multimerizing component. Other components may also be present, or the VEGF trap protein antagonist of the invention may consist essentially, or consist only, of these components. The VEGF trap antagonists used in the method of the invention encompass preferred soluble fusion polypeptides selected from the group consisting of acetylated Flt-1 (1-3)-Fc, Flt-1(1-3_{R→N})-Fc, Flt-1(1-3_{ΔB})-Fc, Flt-1 (2-3_{ΔB})-Fc, Flt-1 (2-3)-Fc, Flt-1D2-VEGFR3D3-FcΔC1(a), Flt-1D2-Flk-1D3-FcΔC1(a), and VEGFR1R2-FcΔC1(a). In a specific and preferred embodiment, the VEGF trap antagonist is VEGFR1R2-FcΔC1(a) (also termed VEGF trap_{R1R2}) having the nucleotide sequence set forth in SEQ ID NO: 1 and the amino acid sequence set forth in SEQ ID NO: 2. The invention encompasses the use of a VEGF trap that is at least 90%, 95%, 98%, or at least 99% homologous with the nucleotide sequence set forth in SEQ ID NO: 1 and/or the amino acid sequence set forth in SEQ ID NO:2.

Administration of the VEGF trap may be by any method known in the art, including subcutaneous, intramuscular, intradermal, intraperitoneal, intravenous, intranasal, or oral routes of administration. In a preferred embodiment, the VEGF trap is administered by subcutaneous injection or intravenous injection. In a more specific embodiment, the VEGF trap is administered by subcutaneous injection.

As described below, the human patient suffering with malignant pleural effusion may also undergo other medical procedures, such as insertion of a pleural catheter or standard chest tube thoracostomy for therapeutic drainage. The method of the invention may be combined with

In one embodiment, the amount of VEGF trap protein administered is in a dosage range between 0.3 mg/kg to 30 mg/kg. In a more specific embodiment, VEGF trap is administered once a day in a range between 0.5 mg/kg to 10 mg/kg. In another embodiment, VEGF trap is administered in a dosage range between 0.3 mg/kg to 30 mg/kg at least once a week. In yet another embodiment, VEGF trap is administered in a dosage range between 0.3 mg/kg to 30 mg/kg at least once a month.

In a second aspect, the invention features a method of treating a human patient suffering malignant pleural effusion related to non-small cell lung cancer, comprising administering a therapeutically effective amount of a vascular endothelial growth factor (VEGF) trap to the human patient. In a preferred embodiment, the VEGF trap administered is a dimer comprised of two fusion polypeptides having the sequence of SEQ ID NO:2. In a further embodiment, the method of the invention is combined with standard therapeutic treatments for obtaining pleural drainage.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims. All applications mentioned herein are specifically incorporated by reference in their entirety.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

### GENERAL DESCRIPTION

Vascular endothelial growth factor/vascular permeability factor (VEGF) was initially identified as a tumor-derived factor capable of increasing vascular permeability. It was subsequently found to be a proliferative factor for endothelial cells. In the embryo, VEGF is absolutely essential for the development of the vasculature. In the adult, VEGF is up-regulated in a variety of normal and pathological processes associated with increased vascular permeability and angiogenesis.

The family of VEGF-related angiogenic growth factors is comprised of VEGF itself (VEGF-A) and the related proteins VEGF-B, -C, -D and E, and placental growth factor (PLGF). In addition, there are at least four different isoforms of VEGF-A. However, as some members of the family have only recently been identified, their biological importance is still poorly understood. The actions of VEGF and its related factors are mediated by a group of three receptor tyrosine kinases, VEGFR1, VEGFR2 and VEGFR3.

Consistent with predictions from animal studies, blockade of VEGF using a humanized monoclonal antibody has emerged reporting promising results in cancer patients, based on preliminary reports from early clinical trials (Bergsland et al. (2000) ASCO Abstract #939). The VEGF trap protein, because of its greater affinity for VEGF and its ability to bind other VEGF family members such as the PIGFs, is a potent and useful anti-cancer therapeutic agent.

Each year, over 160,000 Americans are diagnosed with lung cancer, and approximately 35,000 Americans are diagnosed with a malignant pleural effusion due to lung cancer (Jemal et al. (2002) CA Cancer J. Clin. 52:23-47). The majority of these patients have non-small cell lung cancer (NSCLC). Malignant effusions are comparatively less common in patients with small cell lung cancer, occurring at a rate of less than 3% of all SCLC patients in some series. For patients with NSCLC, a malignant pleural effusion is not considered metastatic disease, but rather T4 disease in the TNM staging classification. Nevertheless, patients with stage IIIB disease who have malignant effusions have worse survival than stage IIIB patients who do not have malignant effusions (16% vs. 45% rate of survival at 5 years in one retrospective study) (Naruke et al. (1997) Chest 112:1710-7). Patients with lung cancer who have malignant pleural effusion are considered to have advanced disease, and are not amenable to surgery or radiation.

Not all malignant pleural effusions contain malignant cells. Various retrospective studies report detection of malignant cells in between 10-50% of suspected malignant effusions (Johnston (1985) Cancer 56:905-9); thus, a pleural effusion does not have to contain malignant cells in order to be considered malignant. In a retrospective study, among patients with NSCLC and pleural effusion, there was no difference in survival time whether the results of fluid cytology testing were positive or negative, provided the latter patients had either bloody and/or exudative fluid that was clinically judged to be the result of the underlying lung cancer (Sugiura et al. (1997) Clin. Cancer Res. 3:47-50). Clinical judgment is necessary to declare an effusion “malignant” in the absence of visible cancer cells, in that bloody effusions can also be caused by traumatic thoracentesis or pulmonary infarction. Approximately 5-10% of lung cancer patients have non-malignant pleural effusions which are due to atelectasis, obstructive pneumonitis, lymphatic or venous obstruction, or pulmonary embolus.

Current Treatment of Malignant Pleural Effusion

Symptomatic malignant pleural effusion from metastatic cancer (i.e., shortness of breath) requires drainage. This can be accomplished by large volume thoracentesis. However, malignant effusions are quick to reaccumulate, and are therefore often treated with a more definitive drainage procedure which involves chest tube thoracostomy followed by instillation of a sclerosing agent, such as talc, bleomycin, or tetracycline, in order to scar the pleura and obliterate the potential space between the parietal and visceral pleura. Patients requiring this procedure are typically admitted to the hospital, and chest tubes are inserted. Generally, the majority of patients with MPE treated with chest tube thoracostomy have advanced non-small cell lung cancer.

An alternative to chest tube thoracostomy and pleurodesis for definitive treatment of MPE involves placement of an ambulatory pleural catheter (Pleur-X™ Catheter, Denver Biomedical, Golden, Colo.). This technique allows for outpatient therapy of MPE, with daily, serial drainage until physiologic pleural scarring occurs, or the cancer is adequately treated with chemotherapy.

A randomized comparison of Pleur-X™ with standard chest tube thoracostomy and doxycycline pleurodesis in 144 patients with recurrent symptomatic MPE has been reported (Putnam et al. (1999) Cancer 86:1992-9). Rate of recurrence of effusion was comparable in both treatment arms, with 21% of doxycycline-treated patients experiencing recurrence of pleural effusion (n=45), as compared to 13% of patients treated with Pleur-X™ (n=99). The degree of symptomatic improvement was nearly identical in both treatment arms. For Pleur-X™ patients, drainage was performed every other day, and catheters were left in place until pleural symphysis was achieved. Pleural symphysis was defined as 3 consecutive drainage attempts without any pleural fluid obtained. Criteria for pleural symphysis is 3 consecutive drainage attempts with ≦50 ml of pleural fluid obtained. In the published study, patients treated with Pleur-X™ achieved pleural symphysis 46% of the time, with a median time to pleural symphysis of 26 days (range 8-223 days). Early complications from the Pleur-X™ catheter included fever (3%), pneumothorax (3%), misplacement of catheter (2%), re-expansion pulmonary edema (1%), and over-sedation during bedside anesthesia (1%). Late complications included cellulitis around the catheter tract (6%), all treated effectively with antibiotics, and none requiring catheter removal. Pain during fluid drainage was reported 7% of the time. Median survival was identical in both treatment arms, approximately 3 months.

A retrospective study of 100 patients treated with Pleur-X™ catheters at one institution documented no mortality related to catheter placement or use, and no morbidity in 81% of patients (Putnam et al. (2000) Ann. Thorac. Surg. 69:369-75). Complications included fluid recurrence due to loculation (8%), catheter malfunction (8%), and infection/empyema (5%). Pleural symphysis was achieved in 21% of patients, with the majority of patients requiring removal of catheter due to complications, or dying with the pleural catheter still in place. The group of patients treated with the Pleur-X™ were retrospectively compared with a group of 68 patients with similar demographics treated with standard chest tube thoracostomy and pleurodesis. The Pleur-X™ group was noted to experience shorter hospitalization time, and lower cost of care, than patients treated with chest tube and pleurodesis. There was no difference in median survival time between the two groups (3.4 months). A smaller retrospective study of 28 patients reported a 42% rate of pleural symphysis, occurring at a median time of 19 days (range 7-96 days) (Pollak et al. (2001) J. Vasc. Interv. Radiol. 12:201-8). A case series of 11 patients with, “Trapped Lung Syndrome,” documented good symptomatic benefit, but no pleural symphysis could be achieved (Pien et al. (2001) Chest 119:1641-6).

Definitions

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding). Efficacy can be measured in conventional ways, depending on the condition to be treated. For cancer therapy, efficacy can, for example, be measured by assessing the time to disease progression, or determining the response rates. Therapeutically effective amount also refers to a target serum concentration, such as a trough serum concentration, that has been shown to be effective in suppressing disease symptoms when maintained for a period of time.

By the term “blocker”, “inhibitor”, or “antagonist” is meant a substance that retards or prevents a chemical or physiological reaction or response. Common blockers or inhibitors include but are not limited to antisense molecules, antibodies, antagonists and their derivatives. More specifically, an example of a VEGF blocker or inhibitor is a VEGF receptor-based antagonist including, for example, an anti-VEGF antibody, or a VEGF trap such as VEGF_{R1R2}-FcΔC1(a) (SEQ ID NOs:1-2). For a complete description of VEGF-receptor based antagonists including VEGF_{R1R2}-FcΔC1(a), see PCT publication WO 00/75319.

The term “package insert” is used to refer to instructions customarily included in commercial packages of therapeutic products, that contain information about the indications, usage, dosage, administration, contraindications and/or warnings concerning the use of such therapeutic products.

The term “intravenous infusion” refers to introduction of a drug into the vein of an animal or human patient over a period of time greater than approximately 5 minutes, preferably between approximately 30 to 90 minutes, although, according to the invention, intravenous infusion is alternatively administered for 10 hours or less.

The term “subcutaneous administration” refers to introduction of a drug under the skin of an animal or human patient, preferable within a pocket between the skin and underlying tissue, by relatively slow, sustained delivery from a drug receptacle. The pocket may be created by pinching or drawing the skin up and away from underlying tissue.

Association of VEGF and Malignant Pleural Effusions (MPE)

Cancer cells cause pleural effusions by invading the pleura, blocking lymphatic drainage of the pleural space, and/or expressing growth factors, and inflammatory cytokines which increase vascular permeability which facilitates capillary leak and further cancer cell invasion (Yano et al. (2000) Am J. Pathol. 157:1893-903). Various signaling molecules and enzymes may contribute to this process, including VEGF, IL-6, IL-8, TGF, metalloproteinases and plasminogen. The importance of VEGF in this process is supported by the discovery of high concentrations of VEGF in malignant effusions and ascites, with levels which are often 10-fold higher than in non-malignant effusions (Kraft et al. (1999) Cancer 85:178-87). VEGF has been implicated in the pathogenesis of MPE from various forms of cancer, including lung cancer, mesothelioma, breast cancer and lymphoma (see, for example, Thickett et al. (1999) Thorax 54:707-10).

A study of 127 patients with various benign and malignant effusions found VEGF levels in pleural fluid to be a reliable marker of malignancy (100% sensitive, 84% specific for malignancy with cut-off value of 2000 pg/ml) (Momi et al. (2002) Respir. med. 96:817-22). In another study, hemorrhagic malignant pleural effusions were found to have significantly higher levels of VEGF in the pleural fluid than non-hemorrhagic MPE, and the malignant cells on pleural biopsy specimens stained reliably for VEGF by IHC using an anti-VEGF antibody (Ishimoto et al. (2002) Oncology 63:70-5). In another case series, both blood and pleural fluid levels of VEGF were significantly higher in patients with lung cancer and pleural effusion compared to patients with benign lung disease (Kishiro et al. (2002) Respirology 7:93-8).

Inhibitors of VEGF have been shown to prevent pleural effusions in animal models (Yano et al. (2000) Clin. Cancer Res. 6:957-65). One study treated cultured endothelial cells with pleural fluid removed from patients with cancer, and documented increased endothelial cell proliferation which could be blocked in vitro by treatment with inhibitors of VEGF (polyclonal anti-VEGF antibodies, and SU5416) (Verheul et al. (2000) Oncologist 5: Suppl. 1:45-50). Another study injected mice with malignant pleural effusion samples, and documented increased vascular permeability which could be blocked in vivo by treatment with inhibitors of VEGF (anti-Flk-1 antibodies) (Zebrowski et al. (1999) Clin. Cancer Res. 5:3364-8).

The VEGF Trap Antagonist

In a preferred embodiment, the VEGF trap antagonist is a receptor-Fc fusion protein consisting of the principal ligand-binding portions of the human VEGFR1 and VEGFR2 receptor extracellular domains fused to the Fc portion of human IgG1. Specifically, the VEGF Trap consists of Ig domain 2 from VEGFR1, which is fused to Ig domain 3 from VEGFR2, which in turn is fused to the Fc domain of IgG1 (SEQ ID NO:2).

In a preferred embodiment, an expression plasmid encoding the VEGF trap is transfected into CHO cells, which secrete VEGF trap into the culture medium. The resulting VEGF trap is a dimeric glycoprotein with a protein molecular weight of 97 kDa and contains ˜15% glycosylation to give a total molecular weight of 115 kDa.

Since the VEGF trap binds its ligands using the binding domains of high-affinity receptors, it has a greater affinity for VEGF than do monoclonal antibodies. The VEGF trap binds VEGF-A (K_{D}=0.5 pM), PLGF1 (K_{D}=1.3 nM), and PLGF2 (K_{D}=50 pM); binding to other VEGF family members has not yet been fully characterized.

Combination Therapies

In numerous embodiments, a VEGF trap may be administered in combination with one or more additional compounds or therapies, including a second VEGF trap molecule, a chemotherapeutic agent, surgery, catheter devices for achieving pleural draining, and radiation. Combination therapy includes administration of a single pharmaceutical dosage formulation which contains a VEGF trap and one or more additional agents; as well as administration of a VEGF trap and one or more additional agent(s) in its own separate pharmaceutical dosage formulation. For example, a VEGF trap and a cytotoxic agent, a chemotherapeutic agent or a growth inhibitory agent can be administered to the patient together in a single dosage composition such as a combined formulation, or each agent can be administered in a separate dosage formulation. Where separate dosage formulations are used, the VEGF-specific fusion protein of the invention and one or more additional agents can be administered concurrently, or at separately staggered times, i.e., sequentially.

The term “cytotoxic agent” as used herein refers to a substance that inhibits or prevents the function of cells and/or causes destruction of cells. The term is intended to include radioactive isotopes (e.g. I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (Cytoxan®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol®, Bristol-Myers Squibb Oncology, Princeton, N.J.) and docetaxel (Taxotere®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, especially a cancer cell either in vitro or in vivo. Examples of growth inhibitory agents include agents that block cell cycle progression (at a place other than S phase), such as agents that induce G1 arrest and M-phase arrest. Classical M-phase blockers include the vincas (vincristine and vinblastine), Taxol®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

Pharmaceutical Compositions

Pharmaceutical compositions useful in the practice of the method of the invention include a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous, subcutaneous, or intramuscular administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention that will be effective in the treatment of diabetes can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Specific Embodiments

Malignant pleural effusion (MPE) is a common complication of advanced non-small cell lung cancer (NSCLC). Symptomatic MPE are generally treated by draining, and ambulatory pleural catheters (Pleur-X™) have been shown to be a viable alternative to standard chest tube thoracostomy for this purpose. While drainage of MPE may provide symptomatic benefit, chemotherapy is the only treatment shown to improve overall survival in patients with advanced NSCLC. Patients with a Pleur-X™ catheter in place may also be treated with chemotherapy, however due to the reported 3-5% incidence of catheter-related infections, non-myelosuppressive chemotherapy would be preferred in this situation. Given the in vivo data suggesting a prominent role for VEGF in the generation of MPE, VEGF trap antagonist described above may have particular efficacy in the treatment of MPE.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Patient Selection and Treatment

Inclusion Criteria: (1) Adult patients with pathologic diagnosis of stage IIIB-IV NSCLC who are eligible for systemic chemotherapy, and also have an MPE which requires therapeutic drainage; (2) Karnofsky performance status of at least 70%; (3) Adequate blood counts, renal and hepatic function; (4) Ability to maintain an ambulatory Pleur-X™ drainage catheter.

Exclusion Criteria: (1) Ongoing chemotherapy with another agent; (2) Prior chemotherapy with an inhibitor of VEGF; (3) Active or untreated brain metastases.

Primary Endpoints: (1) Safety and tolerability; (2) Change in serum and pleural effusion levels of VEGF-A before, and after chemotherapy; (3) Serial gene expression analysis of exfoliated cells isolated from the MPE before, and after chemotherapy.

Other Outcome Variables to be Measured: (1) Volume and rate of pleural fluid collected over time; (2) Time to pleural symphysis/Pleur-X™ catheter removal. Pleural symphysis is defined as 3 consecutive drainages, performed every other day for at least one week, with ≦50 ml of pleural fluid obtained, prompting removal of the catheter; (3) Radiologic response rate; (4) Time to disease progression; (5) Recurrence rate of pleural effusion; (6) Survival.

Protocol Schema: The optimal dose and schedule of VEGF trap is not yet to be determined. Based on the design of current phase I trials, the suggested phase II dose of VEGF Trap is 0.3 to 5 mg/kg IV q2w. Eligible patients must give informed consent prior to enrollment. Patients are admitted to the hospital for placement of a Pleur-X™ ambulatory drainage catheter (Day 0). To prevent re-expansion pulmonary edema, initial drainage volume is limited to 1500 cc of pleural fluid. Additional fluid (up to 1000 cc) may be drained at 8 hour intervals during days 0-1. Beginning on day 3, pleural fluid drainage is performed every other day (qod). To ensure sufficient fluid for analysis, pleural fluid is not drained the day before any subsequent planned fluid collection.

The initial sample of pleural fluid is processed for cytopathology, extraction of tumor-specific RNA, and VEGF-A level as described below. A CT scan of the chest is obtained on Day 0-1, followed by initiation of chemotherapy. VEGF trap is delivered every 2 weeks beginning on day 1. Pleural fluid is collected once per week (day 1, 8, etc.). Exfoliated cells are isolated from the MPE for gene microarray analysis on Day 8. The patient is discharged from the hospital following determination that the Pleur-X™ catheter is functioning properly, and following administration of day 1 chemotherapy. Additional pleural fluid specimens and VEGF levels are obtained 24-72 hours following day 1 chemotherapy, and weekly thereafter.

Patients are removed from the study for any of the following reasons: (1) Intolerable side effects of chemotherapy; (2) Progression of disease as determined by history, physical examination, and/or CT scan; (3) Achievement of pleural symphysis and removal of Pleur-X™ catheter; (4) Any complication related to the Pleur-X™ catheter, and inability to restore a functioning Pleur-X™ catheter. Once removed from study, patients will be treated at the discretion of the treating physician, but will be followed long term for recurrence of pleural effusion, time to treatment progression, and survival.

### Example 2

Standard Analysis of Pleural Fluid

Pleural effusion specimens collected at thoracostomy, or large volume thoracentesis, are analyzed for the presence of malignant cells. Specimens are stored on ice for up to 3 days prior to analysis. Approximately 50 ml of fluid is placed in a conical tube and centrifuged for 10 minutes. Pelleted debris is resuspended in 2 ml of buffered preservative solution, then fixed to a glass slide using either an automated Thin-prep Processor, or manual, double funnel Cytospin device. Resulting slides are stained either using a standard PAP stain, or Diff-Quik stain, then examined under the microscope. Prepared, fixed slides can also be subjected to immunocytochemical staining to aid in diagnosis. Thus, for complete pathologic analysis, a maximum 50 ml of pleural fluid is required. The remainder of the specimen is stored in a refrigerator, and is typically discarded several days later.

Types of cells found in pleural effusions include blood cells (leukocytes and red blood cells), reactive mesothelial cells, and malignant cells. The concentration of malignant cells varies widely between specimens. Immunocytochemistry is routinely used in cytologic analyses to distinguish hyperplastic mesothelial cells from malignant cells, and to assist in the identification of the site of origin of malignant cells (Fetsch et al. (2001) Cancer 93:293-308). A common diagnostic dilemma in lung cancer is the differentiation of adenocarcinoma (NCSLC) from mesothelioma. To make this distinction, pathologists take advantage of several important antigens, including calretinin, which is expressed almost exclusively on mesothelioma, as well as BerEP4, B72.3, and CAl 9-9, which are expressed exclusively on adenocarcinoma. BerEP4 is an antibody prepared by the immunization of mice with cells from the MCF7 breast carcinoma cell line. BerEP4 reacts with two glycoproteins (including Human Epithelial Antigen, HEA) present on the surface and in the cytoplasm of epithelial cells (package insert, Carrpenteria (1998) Dako Corp.). The antibody does not react with mesothelial cells, nerve, glial, muscle or mesenchymal tissue, including lymphoid tissue. In various series, BerEP4 has been shown to react with between 32-96% of all adenocarcinomas tested, with higher rates (>80%) in lung cancer, and low reactivity (0-8%) with mesothelial cells.

The present invention may be embodied in other specific forms without departing from the spirit or essential attributes thereof.

1. A method of treating a human patient suffering from malignant pleural effusion, comprising administering a therapeutically effective amount of a vascular endothelial growth factor (VEGF) antagonist to the human patient.

2. The method of claim 1, wherein the VEGF antagonist is a dimeric protein comprising fusion polypeptides selected from the group consisting of acetylated Flt-1 (1-3)-Fc, Flt-1 (1-3_{R→N})-Fc, Flt-1(1 -3_{ΔB})-Fc, Flt-1 (2-3_{ΔB})-Fc, Flt-1 (2-3)-Fc, Flt-1 D2-VEGFR3D3-FcΔC1(a), Flt-1 D2-Flk-1D3-FcΔC1(a), and VEGF_{R1R2}-FcΔC1(a).

3. The method of claim 2, wherein the fusion polypeptide comprises the amino acid sequence of SEQ ID NO:2.

4. The method of claim 1, wherein administration is subcutaneous, intramuscular, intradermal, intraperitoneal, intravenous, intranasal, or oral.

5. The method of claim 4, wherein administration is by subcutaneous injection.

6. The method of claim 4, wherein administration is by intravenous injection.

7. The method of claim 1, wherein malignant pleural effusion is associated with non-small cell lung cancer.

8. The method of claim 1, wherein the patient undergoes a pleural catheter or standard chest tube thoracostomy for therapeutic drainage.

9. The method of claim 1, wherein the patient is further treated with a chemotherapeutic agent.

10. The method of claim 1, wherein the amount of VEGF antagonist administered is in a dosage range between about 0.3 mg/kg to about 30 mg/kg.

11. The method of claim 10, wherein the dosage range is between 0.5 to 10 mg/kg.

12. The method of claim 11, wherein the dosage range is between 1 to 6 mg/kg.

13. The method of claim 1, wherein the VEGF antagonist is administered once a month.

14. The method of claim 13, wherein the VEGF antagonist is administered at least once a week.

15. The method of claim 14, wherein the VEGF antagonist is administered at least once a day.

16. A method of treating a human patient suffering from malignant pleural effusion associated with non-small cell lung cancer, comprising administering an effective amount of a vascular endothelial growth factor (VEGF) antagonist to the human patient, wherein the VEGF antagonist is a dimeric protein comprising a fusion polypeptide selected from the group consisting of acetylated Flt-1(1-3)-Fc, Flt-1(1-3_{R→N})-Fc, Flt-1(1-3_{ΔB})-Fc, Flt-1(2-3_{ΔB})-Fc, Flt-1(2-3)-Fc, Flt-1D2-VEGFR3D3-FcΔC1(a), Flt-1D2-Flk-1D3-FcΔC1(a), and VEGFR1R2-FcΔC1(a).

17. The method of claim 16, wherein the fusion polypeptide comprises the amino acid sequence of SEQ ID NO:2.

18. A method of treating a human patient suffering from malignant pleural effusion associated with non-small cell lung cancer, comprising administering an effective amount of a vascular endothelial growth factor (VEGF) antagonist to the human patient in conjunction with a chemotherapeutica agent, wherein the VEGF antagonist is a dimeric protein comprising two fusion polypeptides having the sequence of SEQ ID NO:2.
